(19) United States
(12) Patent Application Publication
(10) Pub. No.: US 2023/0304979 A1
(43) Pub. Date: Sep. 28, 2023

(54) INFORMATION PROCESSING DEVICE, INFORMATION PROCESSING METHOD, AND STORAGE MEDIUM

(71) Applicant: NEC Corporation, Minato-ku, Tokyo (JP)

(72) Inventors: Hiromi Shimizu, Tokyo (JP); Shinnosuke Nishimoto, Tokyo (JP); Junko Watanabe, Tokyo (JP); Riki Eto, Tokyo (JP); Noriyuki Tonouchi, Tokyo (JP); So Yamada, Tokyo (JP)

(73) Assignee: NEC Corporation, Minato-ku, Tokyo (JP)

(21) Appl. No.: 18/023,422

(22) PCT Filed: Sep. 2, 2020
(86) PCT No.: PCT/JP2020/033166

§ 371 Date: Feb. 27, 2023

(51) Int. Cl. G06Q 30/0208; G01N 33/00

(52) U.S. Cl. CPC G01N 33/0027; G06Q 30/0208

(57) ABSTRACT

An information processing device is configured to include an acquisition unit, a determination unit, an instruction unit, an instruction unit, and output unit. The acquisition unit is configured to acquire measurement target and measurement environment information, and measurement environment information that a measurer can measure with an odor sensor. The determination unit is configured to determine a measurement target that the measurer should be instructed to measure, based on the measurement target and measurement environment information and the measurement environment information that can be measured, the instruction is configured to instruct the measurer to measure the determined measurement target. The output unit configured to output a reward to the measurer after the acquisition means acquires odor data of the determined measurement target.

### TECHNICAL FIELD

The present invention relates to an information processing device, an information processing method, an information processing method, and a storage medium.

### BACKGROUND ART

PTL 1 provides an information processing device, an information processing method, and a storage medium capable of evaluating a person in charge of labeling in crowdsourcing based on a process of generating a data set as quality improvement of a data set used for machine learning.

### CITATION LIST

### Patent Literature

[PTL 1] WO 2019/069959 A

### SUMMARY OF INVENTION

### Technical Problem

A sensor that acquires odor data has a characteristic that a behavior of a detection value of the sensor changes when a measurement environment such as temperature and humidity changes. Therefore, when labeling the odor data, it is necessary to consider a measurement environment of the odor by the sensor.

However, PTL 1 does not consider an environment in which labeling target data is acquired.

An object of the present invention is to perform crowdsourcing on odor data in consideration of a measurement environment.

### Solution to Problem

An information processing device according to the present invention includes an acquisition means configured to acquire an object to be measured and measurement environment information and information about a measurement environment in which a measurer is allowed to make a measurement with an odor sensor, a determination means configured to determine, based on the object to be measured and the measurement environment information and the measurement-allowed measurement environment information, an object to be measured that the measurer is instructed to measure, an instruction means configured to instruct the measurer to measure the determined object to be measured, and an output means configured to output a reward to the measurer after the acquisition means acquires odor data of the instructed object to be measured.

An information processing method according to the present invention includes acquiring an object to be measured and measurement environment information and information about a measurement environment in which a measurer is allowed to make a measurement with an odor sensor, determining, based on the object to be measured and the measurement environment information and the measurement-allowed measurement environment information, an object to be measured that the measurer is instructed to measure, instructing the measurer to measure the determined object to be measured, and outputting a reward to the measurer when odor data of the instructed object to be measured is acquired.

A storage medium storing a program according to the present invention causes a computer to execute a step of acquiring an object to be measured and measurement environment information and information about a measurement environment in which a measurer is allowed to make a measurement with an odor sensor, a step of determining, based on the object to be measured and the measurement environment information and the measurement-allowed measurement environment information, an object to be measured that the measurer is instructed to measure, a step of instructing the measurer to measure the determined object to be measured, and a step of outputting a reward to the measurer when odor data of the instructed object to be measured is acquired.

### Advantageous Effects of Invention

The present invention can perform crowdsourcing on odor data in consideration of the above-described characteristics.

### BRIEF DESCRIPTION OF DRAWINGS

FIG. 1 is a diagram illustrating a sensor 10 that detect an odor and a time series data obtained by the sensor 10 detecting an odor.

FIG. 2 is a diagram schematically illustrating an information processing system 100.

FIG. 3 is a diagram illustrating a functional configuration of an information processing device 2000 according to the first example embodiment.

FIG. 4 is a diagram illustrating a calculator for implementing the information processing device 2000.

FIG. 5 is a diagram illustrating a flow of processing executed by the information processing device 2000 according to the first example embodiment.

FIG. 6 is a diagram illustrating a screen for receiving a desired object to be measured and measurement environment information.

FIG. 7 is a diagram illustrating a screen for receiving information about a measurement environment in which a measurement can be made with an odor sensor.

FIG. 8 is a diagram illustrating table data stored in a storage unit 2010.

FIG. 9 is a diagram illustrating odor data acquired in the past and stored in a storage unit 2010.

FIG. 10 is a diagram illustrating a frequency distribution of measurement environment information and a distribution of normalized values.

FIG. 11 is a diagram illustrating a screen for displaying an instruction to acquire an object to be measured with a determined reward.

FIG. 12 is a diagram illustrating a screen for acquiring odor data.

FIG. 13 is a diagram illustrating a screen for making a notification of completion of acquisition of odor data.

FIG. 14 is a diagram illustrating a screen for displaying a reward for a contractor.

FIG. 15 is a diagram illustrating a screen for acquiring an image including an object to be measured.

FIG. 16 is a diagram illustrating a screen for receiving acceptance or non-acceptance of an order for a measurement request.

FIG. 17 is a diagram illustrating a screen for requesting a change in a measurement environment.

FIG. 18 is a diagram illustrating a screen for instructing a change in a measurement environment.

FIG. 19 is a diagram illustrating a functional configuration of an information processing device 2000 according to the second example embodiment.

FIG. 20 is a diagram illustrating a flow of processing executed by the information processing device 2000 according to the second example embodiment.

FIG. 21 is a diagram exemplifying a screen displayed by an order receiving terminal 40 when measurement environment information obtained when an odor of the object to be measured is measured does not match information about a measurement environment in which the contractor can make a measurement.

FIG. 22 is a diagram illustrating a functional configuration of an information processing device 2000 according to the third example embodiment.

### EXAMPLE EMBODIMENT

### First Example Embodiment

Hereinafter, the first example embodiment according to the present invention will be described.

### < Sensor >

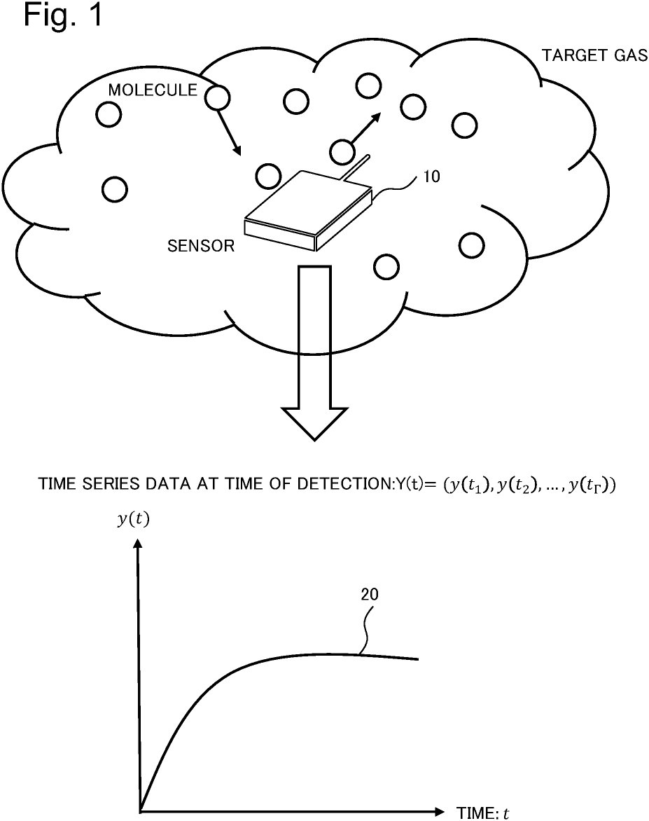

A sensor used in the present example embodiment will be described. FIG. 1 is a diagram illustrating a sensor 10 that detect an odor and a time series data obtained by the sensor 10 detecting an odor. The sensor 10 is a sensor that has a receptor to which a molecule is attached, and in which a detection value changes in response to attachment and detachment of molecules to and from the receptor. The gas sensed by the sensor 10 is referred to as a target gas. The time series data of the detection value output from the sensor 10 is referred to as time series data 20. Here, if necessary, the time series data 20 is also referred to as Y, and the detection value at the time t is also referred to as y(t). Y is a vector in which y(t) is listed.

For example, the sensor 10 may be a membrane-type surface stress sensor (MSS). The MSS has a functional film, as a receptor, to which a molecule adheres, and stress generated in a support member of the functional film changes by attachment and detachment of molecules to and from the functional film. The MSS outputs a detection value based on this change in stress. The sensor 10 is not limited to the MSS, and may be any sensor as long as it outputs a detection value based on a change in a physical quantity related to viscoelasticity and dynamic characteristics (mass, inertia moment, and the like) of a member of the sensor 10, which occurs in response to attachment and detachment of molecules to and from a receptor. Examples of the sensor 10 may include various types of sensors such as a cantilever type sensor, a film type sensor, an optical type sensor, a piezo sensor, and a vibration response sensor.

### < Outline of Present Example Embodiment >

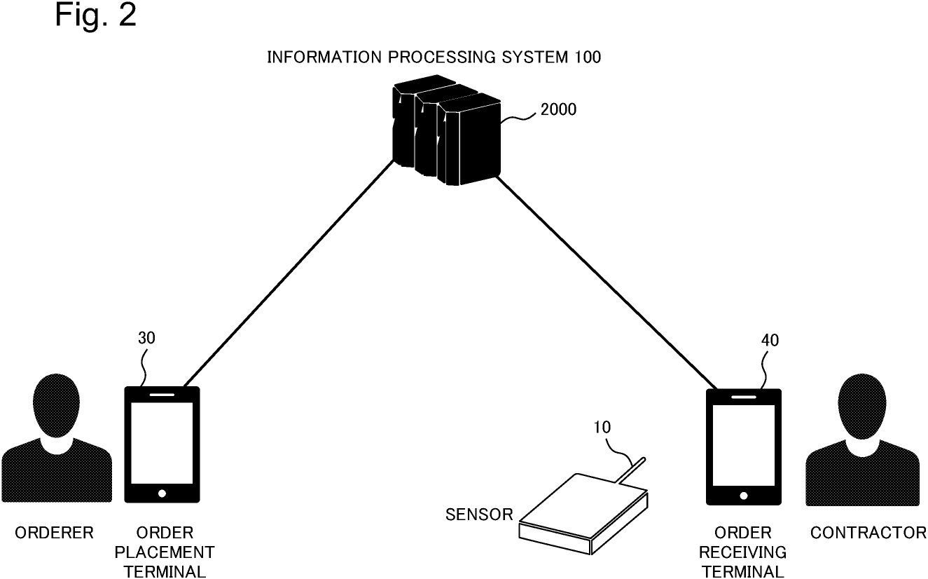

FIG. 2 is a diagram schematically illustrating an information processing system 100. The information processing system 100 performs processing related to crowdsourcing. Crowdsourcing is a service in which an orderer who entrusts a certain task places an order with an unspecified number of contractors for a task. The contractor who is requested for the task selects the task from the requested tasks and receives the order. The contractor obtains a reward by performing the ordered task. Hereinafter, the present example embodiment assumes the following case. That is, the orderer places an order for acquisition of the time series data 20 (hereinafter, the odor data) as a task through crowdsourcing. The contractor receives an order for a task of acquiring odor data using the sensor 10 possessed by the contractor.

The information processing system 100 mainly includes an information processing device 2000, the sensor 10 that acquires odor data by detecting an odor, an order placement terminal 30, and an order receiving terminal 40. The information processing device 2000 and the order placement terminal 30, the information processing device 2000 and the order receiving terminal 40, and the sensor 10 and the order receiving terminal 40 each perform data communication via a communication network or the like. The information processing device 2000 and the sensor 10 may perform data communication via a direct communication network or the like without using the order receiving terminal 40. The information processing device 2000 is equipped with a computer and achieves the function of the information processing device by causing the computer to execute a program. The information processing device 2000 executes the information processing method by the program.

Here, the order placement terminal 30 and the order receiving terminal 40 will be described. The order placement terminal 30 receives an input of a desired object to be measured and measurement environment information from the orderer. The order placement terminal 30 outputs the input desired object to be measured and measurement environment information to the information processing device 2000. The desired object to be measured and measurement environment information will be described later in detail.

The order receiving terminal 40 receives, from the contractor, an input of information about a measurement environment in which a measurement can be made with the sensor 10. The order receiving terminal 40 outputs, to the information processing device 2000, the input information about a measurement environment in which a measurement can be made. Details of the information about a measurement environment in which a measurement can be made with the sensor 10 will be described later.

In the present example embodiment, a case where the contractor is the same as a person (hereinafter, also referred to as “measurer”) who measures the object to be measured using the sensor 10 will be described as an example. However, the measurer and the contractor may be different persons.

In FIG. 2, one sensor 10, one order placement terminal 30, and one order receiving terminal 40 are present, but a plurality of sensors, a plurality of order placement terminals, and a plurality of order receiving terminals may be present.

An outline of processing of the information processing device 2000 will be described. The information processing device 2000 acquires the object to be measured and the measurement environment information desired by the orderer from the order placement terminal 30. The information processing device 2000 acquires, from the order receiving terminal 40, information about a measurement environment in which the contractor can make a measurement with the sensor 10. The information processing device 2000 determines an odor object to be measured based on the acquired information. Details of a method of determining the object to be measured will be described later. The information processing device 2000 outputs information instructing the measurement of the odor of the determined object to be measured to the order receiving terminal 40. The contractor measures the odor of the object to be measured using the sensor 10 based on the instruction displayed by the order receiving terminal 40. The sensor 10 acquires the odor data of the object to be measured to output the data to the order receiving terminal 40. The order receiving terminal 40 outputs the acquired odor data to the information processing device 2000. The information processing device 2000 outputs a reward according to the acquired odor data to the order receiving terminal 40. Details of the processing of the information processing device 2000 will be described later.

### < Example of Functional Configuration of Information Processing device 2000 >

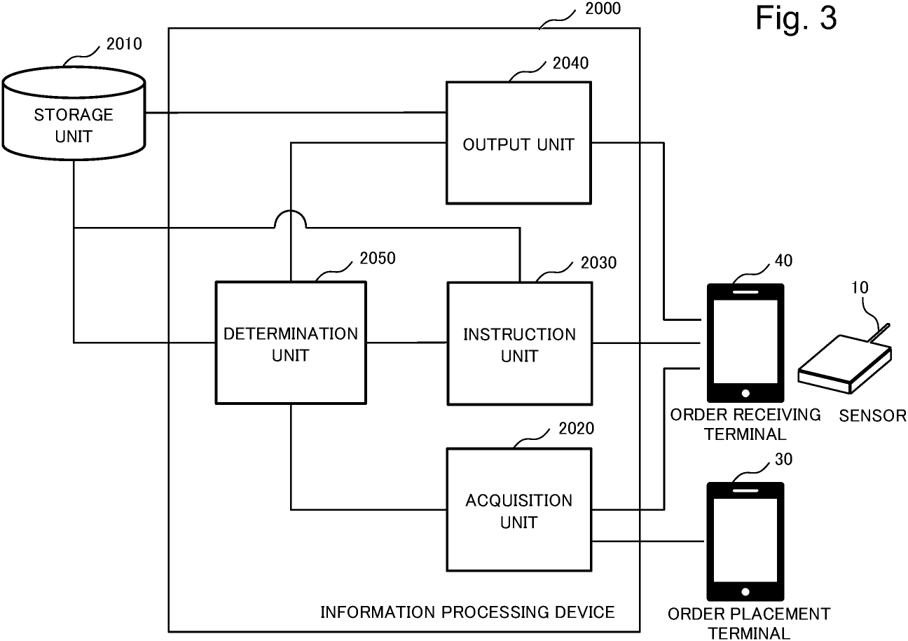

FIG. 3 is a diagram illustrating a functional configuration of the information processing device 2000 according to the first example embodiment. The information processing device 2000 includes an acquisition unit 2020, an instruction unit 2030, an output unit 2040, and a determination unit 2050. The acquisition unit 2020 acquires the object to be measured and the measurement environment information desired by the orderer from the order placement terminal 30 to output the object to be measured and the measurement environment information to the determination unit 2050. The acquisition unit 2020 constitutes an acquisition means configure to acquire, from the order receiving terminal 40, information about a measurement environment in which the contractor can make a measurement with the sensor 10 to output the measurement environment information to the determination unit 2050. The determination unit 2050 constitutes a determination means that determines an object to be measured that the contractor is instructed to measure based on the desired object to be measured and measurement environment information acquired from the acquisition unit 2020 and the information about a measurement environment in which a measurement can be made. The instruction unit 2030 constitutes an instruction means that instructs the contractor to measure the determined object to be measured. The output unit 2040 constitutes an output means that outputs a reward to the contractor who has acquired the odor data of the object to be measured. A method by which the output unit 2040 outputs the reward will be described later. A storage unit 2010 constitutes a storage means that stores odor data to be described later and measurement environment information when the odor data is acquired.

### < Hardware Configuration of Information Processing Device 2000 >

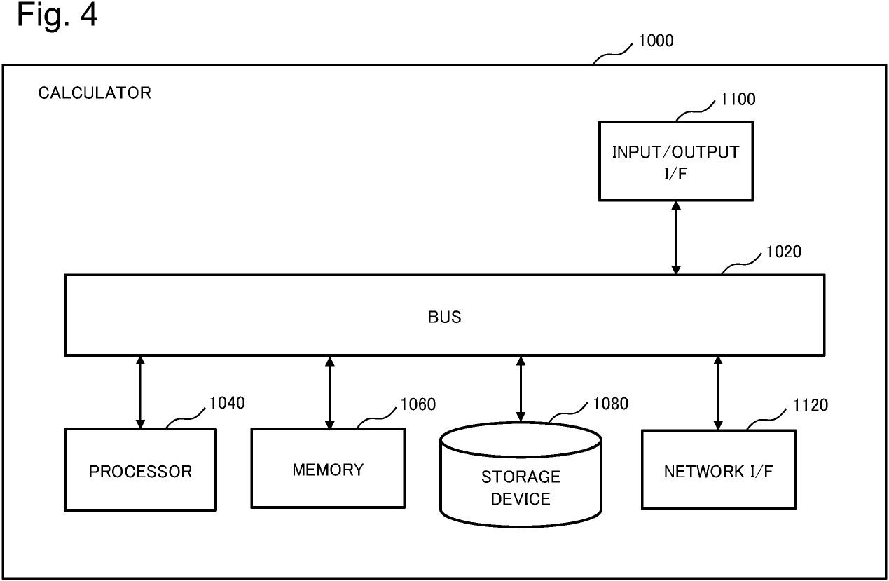

FIG. 4 is a diagram illustrating a calculator for implementing the information processing device 2000 illustrated in FIGS. 2 and 3. A calculator 1000 is an any calculator. For example, the calculator 1000 is a stationary calculator such as a personal computer (PC) or a server machine. Other than the above, for example, the calculator 1000 is a portable calculator such as a smartphone or a tablet terminal. The calculator 1000 may be a dedicated calculator designed to achieve the information processing device 2000, or may be a general-purpose calculator.

The calculator 1000 includes a bus 1020, a processor 1040, a memory 1060, a storage device 1080, an input/output interface 1100, and a network interface 1120. The bus 1020 is a data transmission path for the processor 1040, the memory 1060, the storage device 1080, the input/output interface 1100, and the network interface 1120 to transmit and receive data to and from each other. However, the method of connecting the processor 1040 and the like to each other is not limited to the bus connection.

The processor 1040 is various processors such as a central processing unit (CPU), a graphics processing unit (GPU), and a field-programmable gate array (FPGA). The memory 1060 is a main storage device achieved by using a random access memory (RAM) or the like. The storage device 1080 is an auxiliary storage device achieved using a storage medium such as a hard disk, a solid state drive (SSD), a memory card, or a read only memory (ROM).

The input/output interface 1100 is an interface that connects the calculator 1000 and an input/output device. For example, an input device such as a keyboard and an output device such as a display device are connected to the input/output interface 1100. In addition, for example, the sensor 10 is connected to the input/output interface 1100. However, the sensor 10 is not necessarily directly connected to the calculator 1000. For example, the sensor 10 may store the acquired data in a storage device shared with the calculator 1000.

The network interface 1120 is an interface that connects the calculator 1000 to a communication network. The communication network is, for example, a local area network (LAN) or a wide area network (WAN). A method of connecting the network interface 1120 to the communication network may be wireless connection or wired connection.

The storage device 1080 stores a program module that achieves each functional configuration unit of the information processing device 2000. The processor 1040 reads the program modules into the memory 1060 and executes it, thereby implementing functions related to the program modules.

### < Flow of Processing >

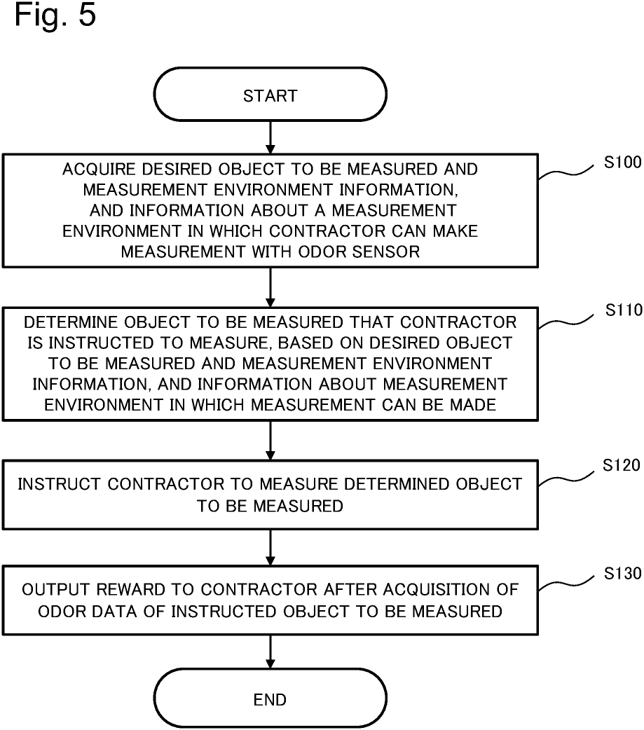

FIG. 5 is a diagram illustrating a flow of processing executed by the information processing device 2000 according to the first example embodiment. The acquisition unit 2020 acquires desired object to be measured and measurement environment information, and information about a measurement environment in which the contractor can make a measurement with the sensor 10 (S100). The determination unit 2050 determines an object to be measured that the contractor is instructed to measure based on the desired object to be measured and measurement environment information and information about a measurement environment in which a measurement can be made (S110). The instruction unit 2030 instructs the contractor to measure the determined object to be measured (S120). After acquiring the odor data of the instructed object to be measured, the output unit 2040 outputs a reward to the contractor (S130).

### < Object to Be Measured and Measurement Environment information Desired by Orderer >

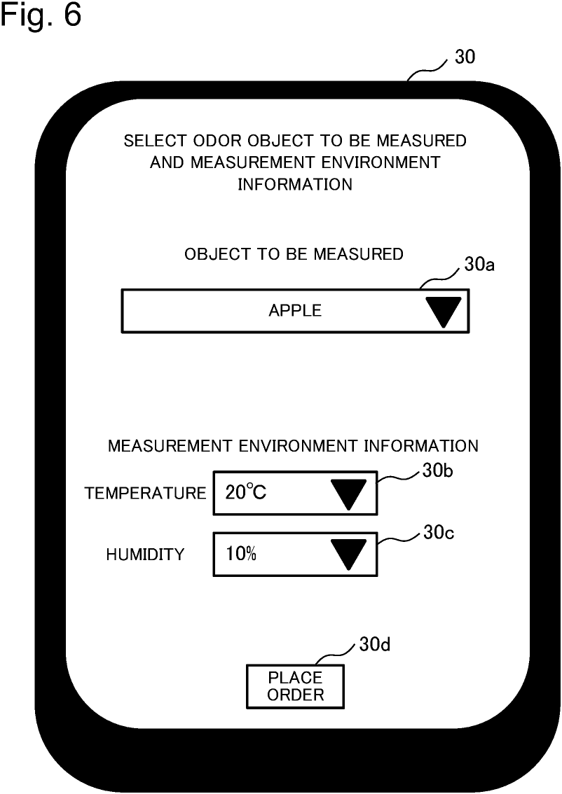

Desired object to be measured and measurement environment information acquired by the acquisition unit 2020 will be described with reference to FIG. 6. FIG. 6 is a diagram illustrating a screen for receiving desired object to be measured and measurement environment information. Such a screen is displayed on the order placement terminal 30. The screen illustrated in FIG. 6 includes, for example, a message urging an input of the odor object to be measured that the orderer who places an order for the measurement of the odor data desires and the measurement environment information, an object to be measured selection field 30a, a measurement environment information (temperature) selection field 30b, a measurement environment information (humidity) selection field 30c, and an order button 30d.

The object to be measured selection field 30a is a selection field for designating an object to be measured desired by the orderer. Here, as an example, the object to be measured selection field 30a is a pull-down menu type selection field, and “apple” is selected in FIG. 6.

The measurement environment information (temperature) selection field 30b is a selection field for designating measurement environment information related to temperature desired by the orderer. Here, as an example, the measurement environment information (temperature) selection field 30b is a pull-down menu type selection field, and “20° C.” is selected in FIG. 6.

The measurement environment information (humidity) selection field 30c is a selection field for designating measurement environment information about humidity desired by the orderer. Here, as an example, the measurement environment information (humidity) selection field 30c is a pull-down menu type selection field, and “10%” is selected in FIG. 6.

The order button 30d is a button for instructing to proceed with the order based on the object to be measured and the measurement environment information input by the orderer. For example, when the orderer presses the order button 30d, the acquisition unit 2020 acquires the object to be measured and the measurement environment information input by the orderer.

In FIG. 6, the measurement environment information (temperature) selection field 30b and the measurement environment information (humidity) selection field 30c are not limited to pull-down type selection fields. Each selection field can receive an input of measurement environment information from the orderer via a keyboard or the like, for example.

### < Type of Measurement Environment Information >

In FIG. 6, the acquisition unit 2020 acquires temperature and humidity as measurement environment information. However, the measurement environment information acquired by the acquisition unit 2020 is not limited to the temperature and humidity, and it is possible to acquire information about the environment at the time of measuring the odor. For example, the measurement environment that can be acquired by the acquisition unit 2020 includes the temperature, humidity, and sampling cycle of the environment in which the sensor 10 is installed.

The sampling cycle indicates an interval for measuring the odor, and is expressed as Δt [s] or a sampling frequency [Hz] using an inverse of Δt [s]. For example, the sampling cycle is 0.1 [s], 0.01 [s], or the like.

When the odor is measured by alternately injecting the sample gas and the purge gas to the sensor 10, the sample gas and the purge gas injection time may be set as the sampling cycle. Here, the sample gas is the target gas in FIG. 1. The purge gas is a gas (for example, nitrogen) for removing the target gas attached to the sensor 10. For example, the sensor 10 can measure data by injecting the sample gas for 5 seconds and the purge gas for 5 seconds.

The measurement environment such as the temperature, humidity, and sampling cycle described above may be acquired by, for example, a meter provided inside or outside the sensor 10, or may be input from the user through the order placement terminal 30.

Examples of other measurement environments include information about the distance between the object to be measured and the sensor 10, the type of purge gas, the carrier gas, the type of sensor (for example, the sensor ID), the season at the time of measurement, the atmospheric pressure at the time of measurement, the atmosphere at the time of measurement (for example, CO₂ concentration), and the contractor. The carrier gas is a gas injected simultaneously with the odor of the object to be measured, and for example, nitrogen or the atmosphere is used. The sample gas is a mixture of the carrier gas and the odor of the object to be measured.

The above-described temperature and humidity may be acquired from the object to be measured, the carrier gas, the purge gas, the sensor 10 itself, the atmosphere around the sensor 10, the sensor 10, or a setting value of a device that controls the sensor 10.

### < Information About a Measurement Environment in Which contractor can make a measurement with sensor 10 >

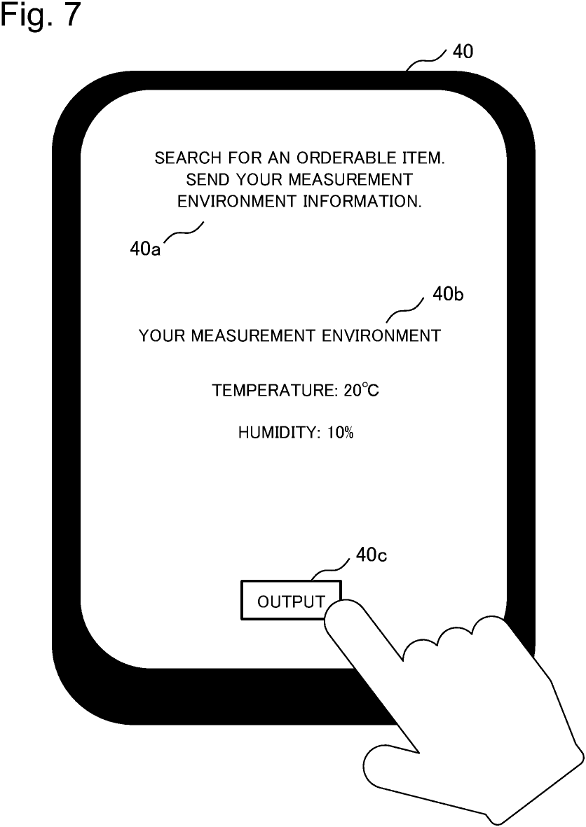

With reference to FIG. 7, information, acquired by the acquisition unit 2020, about a measurement environment in which the contractor can make a measurement with the sensor 10 will be described. FIG. 7 is a diagram illustrating a screen for receiving information about a measurement environment in which a measurement can be made with the sensor 10. Such a screen is displayed on the order receiving terminal 40. The screen illustrated in FIG. 7 includes, for example, a message 40a urging an output of information about a measurement environment in which the contractor can make a measurement with the sensor 10, information 40b about a measurement environment in which the contractor can make a measurement with the sensor 10, and an output button 40c.

As an example of the information 40b about a measurement environment in which the contractor can make a measurement with the sensor 10, the screen illustrated in FIG. 7 displays “temperature: 20° C.” and “humidity: 10%”. The measurement environment such as temperature and humidity may be acquired by, for example, a meter such as a hygrometer or a thermometer provided inside or outside the sensor 10, or an input may be received from the contractor through a keyboard or the like.

The output button 40c is a button for instructing to output information about a measurement environment in which the contractor can make a measurement with the sensor 10 to the information processing device 2000. For example, when the contractor presses the output button 40c, the acquisition unit 2020 acquires information about a measurement environment in which the contractor can make a measurement with the sensor 10.

The screen illustrated in FIG. 7 outputs the humidity and the temperature as the information about a measurement environment in which a measurement can be made with the sensor 10, but the measurement environment information output by the screen illustrated in FIG. 7 is not limited to the temperature or the humidity. The measurement environment output by the screen illustrated in FIG. 7 may be any information about the environment when measuring the odor as in the case of the object to be measured and the measurement environment information desired by the orderer. For example, the screen illustrated in FIG. 7 may receive, as information about a measurement environment in which a measurement can be made with the sensor 10, a sampling cycle, the distance between the object to be measured and the sensor 10, the type of purge gas, the carrier gas, the type of sensor (for example, the sensor ID), the season at the time of measurement, the atmospheric pressure at the time of measurement, the atmosphere at the time of measurement (for example, CO₂ concentration), and information about the contractor (place of residence, body temperature, etc.).

### < Operation of Determination Unit 2050 >

The operation of the determination unit 2050 will be described. First, the determination unit 2050 performs matching between the contractor and the orderer based on the desired object to be measured and measurement environment information acquired from the acquisition unit 2020 and the information about a measurement environment in which the contractor can make a measurement with the sensor 10. The determination unit 2050 may perform matching between the orderer and the contractor in such a way that the desired measurement environment information matches the information about a measurement environment in which the contractor can make a measurement with the sensor 10. When the determination unit 2050 performs matching, in a case where the difference between the desired measurement environment information and the information about a measurement environment in which a measurement can be made is within a predetermined threshold value, matching between the orderer and the contractor may be established.

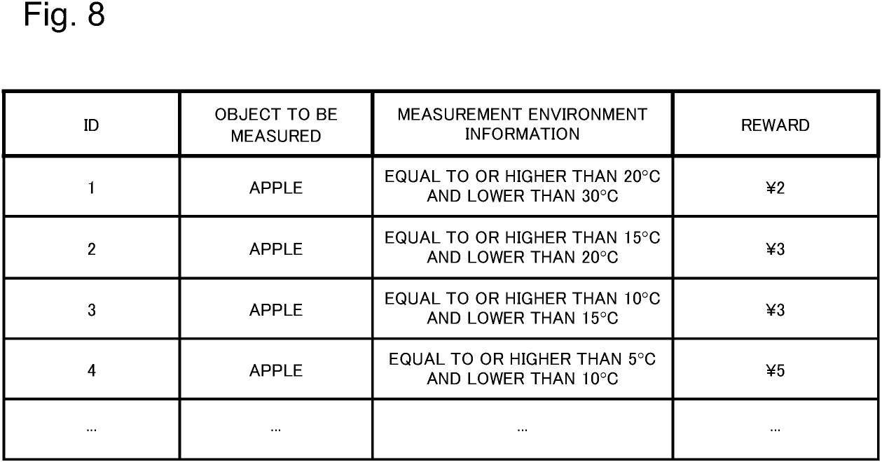

Next, the determination unit 2050 determines a reward to the contractor with which matching is established. An operation in which the determination unit 2050 determines the reward to be paid to the contractor who has measured the object to be measured will be described with reference to FIG. 8. FIG. 8 is a diagram illustrating table data stored in the storage unit 2010. Each record in FIG. 8 includes, for example, an ID for identifying each record, an object to be measured, measurement environment information, and a reward.

First, the determination unit 2050 identifies a record including the object to be measured and the measurement environment information desired by the orderer acquired from the acquisition unit 2020. For example, it is assumed that the determination unit 2050 acquires, from the acquisition unit 2020, “apple” as an object to be measured desired by the orderer and acquires “temperature: 20° C.” as measurement environment information desired by the orderer. In this case, the determination unit 2050 identifies a record with an ID of “1” in which the object to be measured is “apple” and the measurement environment information is “equal to or higher than 20° C. and lower than 30° C.”.

Next, the determination unit 2050 determines a reward included in the identified record as a reward to be paid to the contractor who has measured the object to be measured. For example, in a case where the identified record is a record with an ID “1”, the determination unit 2050 determines the reward as“¥2”.

Then, the determination unit 2050 outputs information (for example, the ID of the order receiving terminal owned by the contractor) of the determined contractor, the object to be measured desired by the orderer, and the determined reward to the instruction unit 2030.

In a case where the determination unit 2050 determines that the identified record does not include information about an environment in which a measurement can be made, the determination unit 2050 outputs information indicating that there is no item that can be requested to the instruction unit 2030.

In the above description, the method in which the determination unit 2050 determines the object to be measured that the contractor is instructed to measure and the reward based on the table data illustrated in FIG. 8 is described. However, the determination unit 2050 may determine the reward without using the table data illustrated in FIG. 8.

Here, as an example in which the determination unit 2050 determines a reward without referring to the table data illustrated in FIG. 8, a method of determining a reward based on odor data acquired in the past will be described.

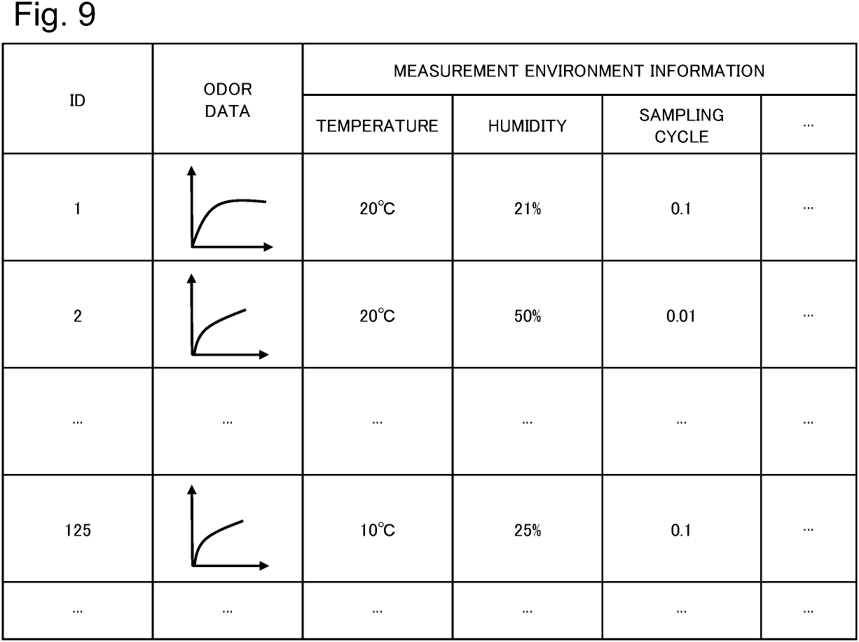

FIG. 9 is a diagram illustrating odor data acquired in the past and stored in the storage unit 2010. Each record in FIG. 9 includes, for example, at least an ID for identifying odor data, odor data obtained by the sensor 10 detecting an odor, and measurement environment information. In FIG. 9, the measurement environment information includes the temperature, the humidity, and the sampling cycle, but the measurement environment information is not limited to the temperature and the humidity, and can acquire information on the environment when the odor is measured. For example, the measurement environment information that the record of FIG. 9 can have includes information on the distance between the object to be measured and the sensor 10, the type of purge gas, the carrier gas, the type of sensor (for example, the sensor ID), the season at the time of measurement, the atmospheric pressure at the time of measurement, the atmosphere at the time of measurement (for example, CO₂ concentration), and the contractor.

The determination unit 2050 acquires at least one or more pieces of previously acquired odor data from the storage unit 2010. Next, the determination unit 2050 may calculate a statistic of the measurement environment information related to each acquired odor data and determine the reward based on the statistic.

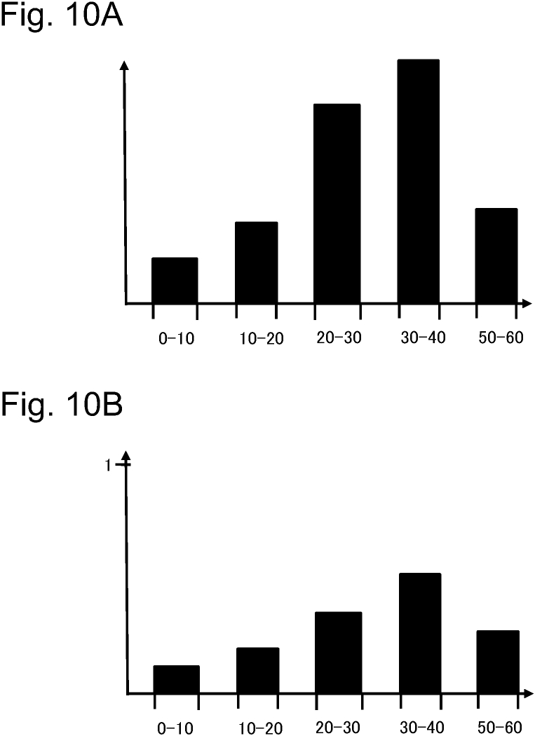

Here, an example of a method in which the determination unit 2050 determines the reward based on the statistic will be described with reference to FIG. 10. FIG. 10 is a diagram illustrating a frequency distribution of measurement environment information and a distribution of normalized values. In the frequency distribution illustrated in FIG. 10(A), values of measurement environment information (for example, temperature) in which previously acquired odor data was measured are given along the horizontal axis at predetermined intervals (for example, every 10° C.). The values on the vertical axis indicates the appearance frequency of pieces of measurement environment information along the horizontal axis.

Next, as illustrated in FIG. 10(B), the determination unit 2050 normalizes the calculated appearance frequency distribution to convert the frequency of pieces of measurement environment information along the horizontal axis into a normalized value expressed by a continuous value from 0 to 1. Then, for example, the determination unit 2050 may determine the reward for each piece of measurement environment information based on the normalized value for each piece of measurement environment information along the horizontal axis with respect to a predetermined budget.

Here, an example of a method in which the determination unit 2050 determines the reward for each piece of measurement environment information based on the normalized value will be described. First, the determination unit 2050 calculates the reciprocal of the normalized value of each piece of measurement environment information. For example, it is assumed that a normalized value of each piece of measurement environment information illustrated in FIG. 10(B) is as follows. “0-10” : 0.05, “10-20” : 0.1, “20-30” : 0.3, “30-40” : 0.4, “40-50” : 0.15. In this case, the determination unit 2050 calculates the reciprocal of the normalized value of each piece of measurement environment information as follows. “0-10” : 20, “10-20” : 10, “20-30” : 3.3, “30-40” : 2.5, “40-50” : 6.67.

Next, the determination unit 2050 calculates a ratio of the reciprocal of the normalized value. For example, the determination unit 2050 calculates the ratio as follows using the above-described reciprocal. “0-10” : 0.47, “10-20” : 0.23, “20-30” : 0.1, “30-40” : 0.05, “40-50” : 0.15.

Then, the determination unit 2050 can determine the reward for each piece of measurement environment information by multiplying the predetermined budget by the ratio of the calculated reciprocal. For example, a case where the determination unit 2050 acquires 500 yen as the predetermined budget from the acquisition unit will be described. For example, the determination unit 2050 calculates 500 yen × 0.1 = 50 yen as a reward for the measurement environment information “20-30”.

As described above, the determination unit 2050 can allocate a high reward to the measurement environment information (that is, valuable measurement environment information) with a low frequency by using the ratio of the reciprocal of the normalized value.

The acquisition unit 2020 may acquire the predetermined budget from the orderer through the order placement terminal 30. In this case, the screen illustrated in FIG. 6 may display a selection field for specifying the budget. For example, the selection field for specifying the budget can receive specification of the upper limit of the amount of money from the orderer via a keyboard or the like. When the orderer presses selection of the order button 30d after inputting the budget, the acquisition unit 2020 acquires the budget input by the orderer.

The statistic of the measurement environment information is not limited to the normalized value illustrated in FIG. 10(B), and may be any value as long as the priority order of the measurement environment information can be determined. For example, the determination unit 2050 may determine the priority order of the measurement environment information using at least one of a known statistical method, a known mathematical optimization method, or a known machine learning method, and determine the reward using the determined priority order.

In FIGS. 10(A) and (B), values of temperature are given along the horizontal axis as the measurement environment information. However, the given measurement environment information is not limited to one type, and a combination of two or more types of measurement environment information may be given.

### < Operation Example of Instruction Unit 2030 >

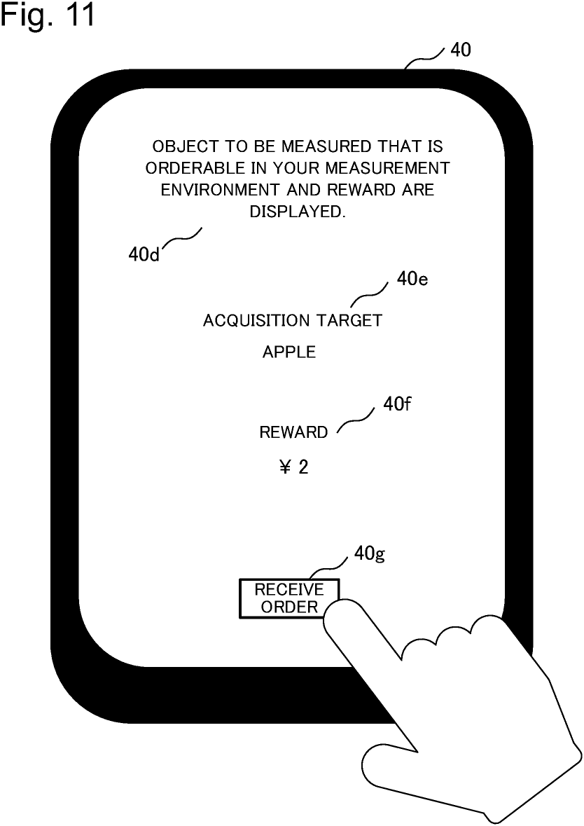

An operation of the instruction unit 2030 will be described with reference to FIG. 11. The instruction unit 2030 outputs an instruction to acquire the object to be measured with the determined reward to the order receiving terminal 40 based on the information (for example, the ID of the order receiving terminal owned by the contractor) of the contractor acquired from the determination unit 2050, the object to be measured desired by the orderer, and the determined reward. The order receiving terminal 40 displays the instruction output from the instruction unit 2030 on the screen. FIG. 11 is a diagram illustrating a screen for displaying an instruction to acquire an object to be measured with a determined reward. Such a screen is displayed on the order receiving terminal 40. The screen illustrated in FIG. 11 includes a message 40d indicating an output of information about a measurement environment in which the contractor can make a measurement with the sensor 10, is output, an object to be measured 40e, a reward 40f, and an order acceptance button 40 g.

As an example of the object to be measured 40e and the reward 40f, the screen illustrated in FIG. 11 displays “apple” as the object to be measured and “¥2” as the reward.

An order acceptance button 40 g is a button for instructing to notify the information processing device 2000 that the contractor has received an order for a measurement request. When the contractor presses the order acceptance button 40 g, the screen illustrated in FIG. 11 transitions to a screen for acquiring odor data to be described later.

The order receiving terminal 40 may display an order non-acceptance button (not illustrated). When the contractor presses the order non-acceptance button, the screen illustrated in FIG. 11 ends the screen display.

The screen illustrated in FIG. 11 displays only “apple” as the object to be measured. However, the screen illustrated in FIG. 11 may display a plurality of objects to be measured and receive acceptance or non-acceptance of an order for each object to be measured.

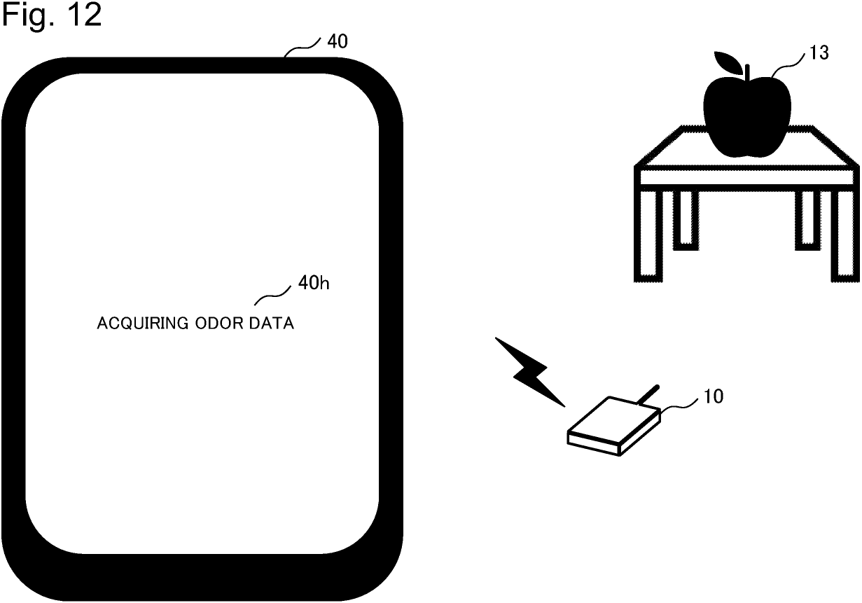

A screen for acquiring the odor data, which is displayed when the order receiving terminal 40 receives the selection of the order acceptance button 40 g, will be described. FIG. 12 is a diagram illustrating an example of a screen for acquiring odor data. Such a screen is displayed on the order receiving terminal 40. As illustrated in FIG. 12, the contractor uses the sensor 10 to acquire odor data of an object to be measured 13. While the contractor acquires the odor data, the screen illustrated in FIG. 12 displays a message 40h indicating that the odor data is being acquired. When the order receiving terminal 40 detects the acquisition of the odor data, the screen illustrated in FIG. 12 transitions to a screen for making a notification of completion of the acquisition of the odor data to be described later.

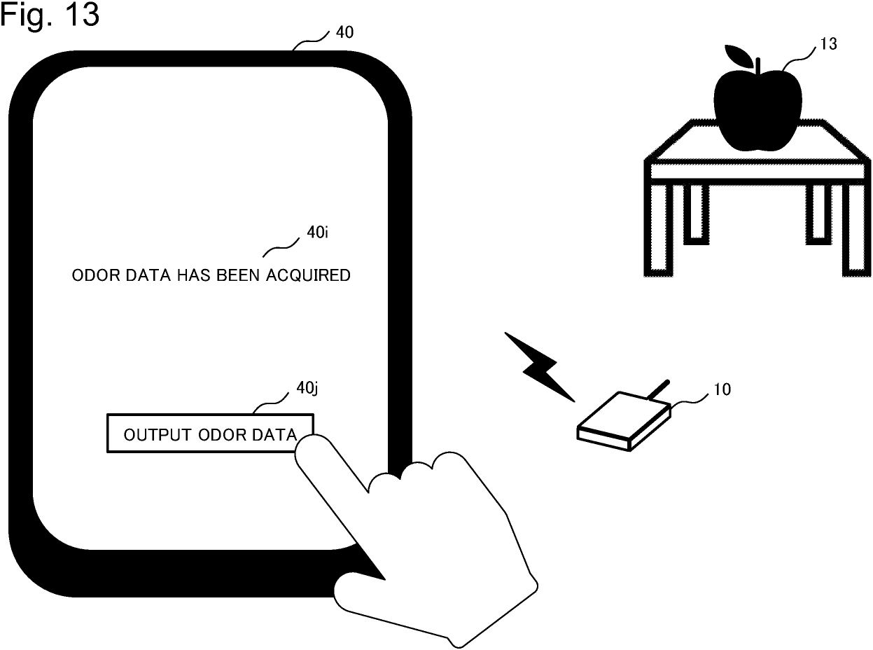

FIG. 13 is a diagram illustrating an example of a screen for making a notification of completion of acquisition of odor data. Such a screen is displayed on the order receiving terminal 40. The screen illustrated in FIG. 13 includes a message 40i indicating that the acquisition of the odor data has been completed and an output button 40j.

The output button 40j is a button for instructing to output, to the information processing device 2000, the odor data the contractor has acquired with the odor sensor. When the contractor presses the output button 40j, the acquisition unit 2020 acquires the odor data acquired by the sensor 10.

### < Operation Example of Output Unit 2040 >

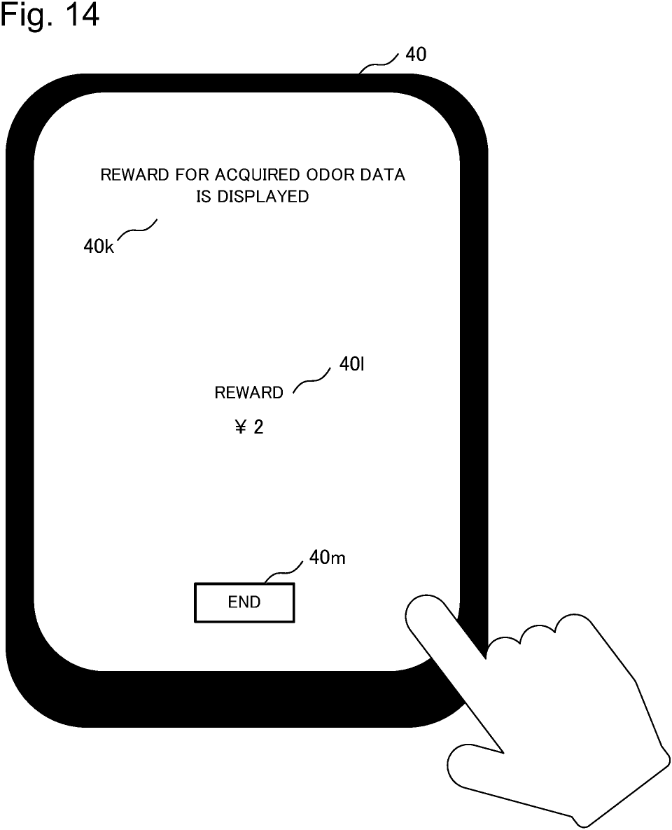

The operation of the output unit 2040 will be described with reference to FIG. 14. FIG. 14 is a diagram illustrating a screen for displaying a reward to a contractor. Such a screen is displayed on the order receiving terminal 40. The screen illustrated in FIG. 14 includes a message 40k indicating that a reward for the acquired odor data is displayed, a reward 401 (for example, “¥2”), and an end button 40 m.

The end button 40 m is a button for instructing to notify the information processing device 2000 that the contractor has completed the measurement request. When the contractor presses the end button 40 m, the output unit 2040 outputs the amount of money displayed by the reward 401. As a method by which the output unit 2040 outputs the reward 401, for example, the output unit 2040 transfers an amount of the reward 401 to the bank account of the contractor. Other than the above, for example, the output unit 2040 notifies, by e-mail or the like, the administrator of the information processing device 2000 of an instruction to pay the reward 401. The administrator performs a payment procedure of the reward 401 to the contractor based on the instruction of which the output unit 2040 makes a notification.

### < Action and Effect >

As described above, the information processing device 2000 according to the present example embodiment performs crowdsourcing on the odor data based on the object to be measured and the measurement environment information desired by the orderer and the information about a measurement environment in which the contractor can make a measurement with the sensor 10. Therefore, the information processing device 2000 according to the present example embodiment can perform crowdsourcing in consideration of the characteristic that the behavior of the odor data changes when the measurement environment such as temperature and humidity changes.

### First Modification

The first modification of the first example embodiment will be described. In the first modification, the determination unit 2050 determines an object to be measured that the contractor is instructed to measure based on the image. Specifically, the acquisition unit 2020 acquires an image including a measurable object to be measured. The determination unit 2050 determines an object to be measured that the contractor is instructed to measure based on the object to be measured and the measurement environment information desired by the orderer and the acquired image and the information about a measurement environment in which the contractor can make a measurement with the sensor 10.

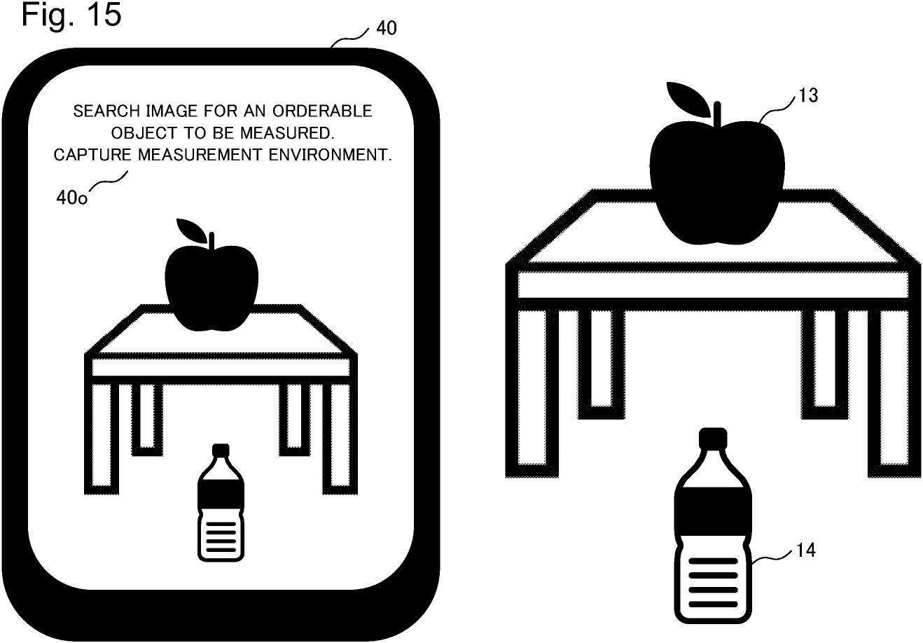

An image including an object to be measured that can be measured with the sensor 10 and acquired by the acquisition unit 2020 will be described with reference to FIG. 15. FIG. 15 is a diagram illustrating an example of a screen for acquiring an image including an object to be measured. Such a screen is displayed on the order receiving terminal 40. The screen illustrated in FIG. 15 includes a message 40o indicating an instruction to capture the measurement environment. The contractor captures an image of the measurement environment including the object to be measured 13 using a camera installed in the order receiving terminal 40. Here, the captured image may include an object 14 other than the object to be measured.

The acquisition unit 2020 acquires the captured image to output the image to the determination unit 2050. The determination unit 2050 determines whether the acquired image includes the object to be measured desired by the orderer using a known image recognition technique. In a case where it is determined that the object to be measured desired by the orderer is included in the image, the screen illustrated in FIG. 15 transitions to a screen for receiving acceptance or non-acceptance of an order for a measurement request to be described later. In a case where it is determined that the object to be measured desired by the orderer is not included in the image, the screen illustrated in FIG. 15 transitions to a screen illustrated in FIG. 11 for receiving acceptance or non-acceptance of an order for the measurement request.

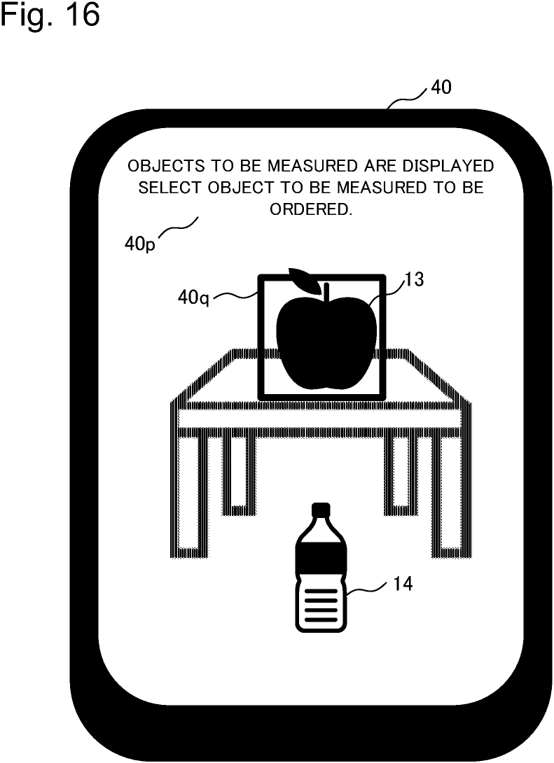

An operation of the instruction unit 2030 using an image will be described with reference to FIG. 16. FIG. 16 is a diagram illustrating a screen for receiving acceptance or non-acceptance of an order for a measurement request. Such a screen is displayed on the order receiving terminal 40. The screen illustrated in FIG. 16 includes a message 40p for selecting an ordered item and an orderable item 40q on the image captured by the contractor.

The orderable item 40q is an object to be measured which is included in the image and that can be ordered. For example, the screen illustrated in FIG. 16 displays the object to be measured 13 as the orderable item 40q.

When the contractor presses the orderable item 40q, the screen illustrated in FIG. 16 transitions to a screen for acquiring odor data illustrated in FIG. 12.

### < Action and Effect >

In the first modification of the first example embodiment, the determination unit 2050 determines the object to be measured based on the image including the object to be measured that can be ordered by the contractor. The contractor does not need to separately prepare an instructed object to be measured, and may measure the object to be measured included in the image. Therefore, the first modification of the first example embodiment can efficiently measure the odor of the object to be measured.

### Second Modification

The second modification of the first example embodiment will be described. In the second modification of the first example embodiment, it is possible to request the measurement of the odor when the measurement environment is changed with respect to the object whose odor was measured by the contractor.

Specifically, the instruction unit 2030 refers to the table data illustrated in FIG. 8 stored in the storage unit 2010, and determines whether there is measurement environment information (hereinafter, it is described as “changeable measurement environment information”) that is different from the information about a measurement environment in which the object was measured and for which a reward is set for the object to be measured whose odor was measured by the contractor. When it is determined that there is the changeable measurement environment information, the instruction unit 2030 outputs, to the order receiving terminal 40, the changeable measurement environment information, a reward related to the changeable measurement environment information, and an instruction to change the measurement environment.

When determining that there is no changeable measurement environment information, the instruction unit 2030 outputs, to the order receiving terminal 40, information indicating that there is no changeable measurement environment information.

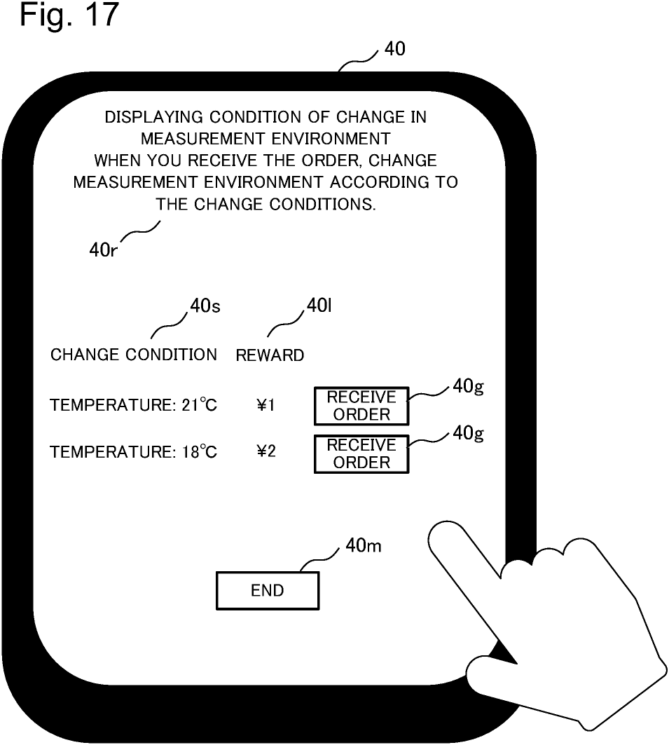

FIG. 17 is a diagram illustrating a screen for requesting a change in the measurement environment. Such a screen is displayed on the order receiving terminal 40. The screen illustrated in FIG. 17 includes an instruction 40r to change the measurement environment, a change condition 40s, a reward 401, an order acceptance button 40 g, and an end button 40 m.

The change condition 40s indicates changeable measurement environment information. As an example of the change condition 40s, the screen illustrated in FIG. 17 illustrates “temperature 21° C.” and “temperature 18° C.”.

The reward 401 indicates a reward related to the changeable measurement environment information. As an example of the reward 401, the screen illustrated in FIG. 17 indicates that the reward is “¥1” when odor data is acquired at “temperature 21° C.”, and the reward is “¥2” when odor data is acquired at “temperature 18° C.”.

An order acceptance button 40 g is a button for instructing to notify the information processing device 2000 that the contractor has received an order for a measurement request. When the contractor presses the order acceptance button, the screen illustrated in FIG. 17 transitions to a screen for acquiring odor data illustrated in FIG. 12.

The output unit 2040 outputs a reward determined according to the measurement environment when detecting the acquisition of the odor data in the changed measurement environment.

The end button 40 m is a button for instructing to notify the information processing device 2000 that the contractor does not change the measurement environment. When the contractor presses the end button 40 m, the screen 17 ends.

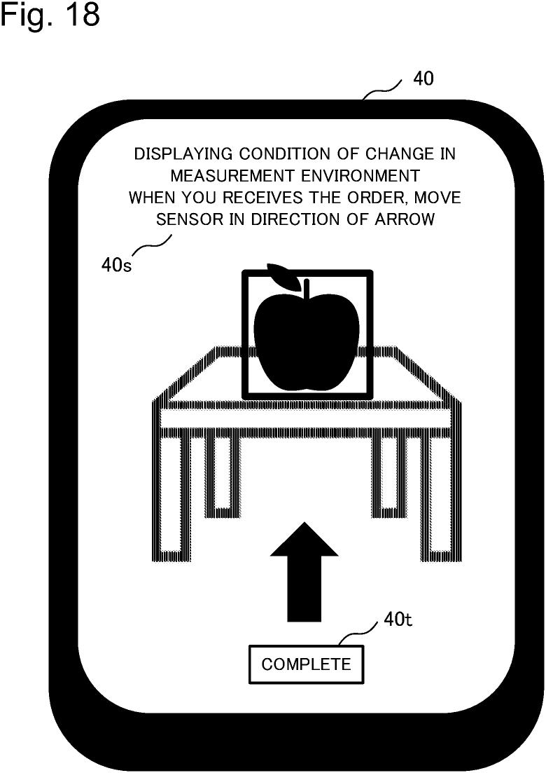

The instruction unit 2030 may instruct a change in the measurement environment based on the image acquired by the acquisition unit 2020. Here, an example of an operation in which the instruction unit 2030 instructs a change in the measurement environment using the image will be described with reference to FIG. 18. FIG. 18 is a diagram illustrating a screen for instructing a change in a measurement environment. Such a screen is displayed on the order receiving terminal 40. The screen illustrated in FIG. 18 includes a message 40s instructing a change in the measurement environment information and a completion button 40t. The screen illustrated in FIG. 18 displays an instruction to change the position of the sensor 10 as the measurement environment. The contractor changes the position of the sensor based on the displayed instruction, thereby changing the measurement environment as the distance between the object to be measured 13 and the sensor 10.

Here, the reason why the distance between the object to be measured 13 and the sensor 10 is one of the measurement environments that affect the odor data will be described. The sensor 10 outputs a change in stress generated in the support member of the functional film due to attachment and detachment of odor molecules to and from the functional film as odor data. When the distance between the object to be measured 13 and the sensor 10 changes, the amount of odor molecules attached to the functional film changes even in the same object to be measured. From the above reasons, it can be said that the distance between the object to be measured 13 and the sensor 10 is one of the measurement environments that affect the odor data.

The completion button 40t is a button for instructing to notify the information processing device 2000 that the contractor has completed the change in the measurement environment. When the contractor presses the completion button 40t, the screen illustrated in FIG. 18 transitions to a screen for acquiring the odor data illustrated in FIG. 12.

### < Action and Effect >

In the second modification of the first example embodiment, the instruction unit 2030 instructs a change in the measurement environment, and the output unit 2040 can output a reward to the contractor when the acquisition unit 2020 acquires the odor data of the object to be measured in the changed measurement environment. Therefore, in the second modification of the first example embodiment, it is possible to efficiently acquire the odor data of the same object to be measured in a plurality of different environments.

### Second Example Embodiment

Hereinafter, the second example embodiment according to the present invention will be described. First, problems assumed by the second example embodiment will be described. When the contractor measures the odor using the sensor 10, the information about a measurement environment, input by the contractor via the order receiving terminal 40, in which a measurement can be made may be different from the measurement environment information when actually measured using the sensor 10. For example, the time point (1) when the information about a measurement environment in which the contractor can make a measurement is input and the time point (2) when the odor of the object to be measured is actually measured are distant, the measurement environment information such as temperature and humidity may change between the time point (1) and the time point (2). In order to obtain a high reward, it is also conceivable that the contractor inputs false information as the information about a measurement environment in which a measurement can be made. In such a case, the information processing device 2000 may not be able to accurately acquire the odor data in the desired measurement environment information.

A second example embodiment according to the present invention outputs a reward when it is determined that odor data is measured in a measurement environment desired by an orderer.

### < Example of Functional Configuration of Information Processing Device 2000 >

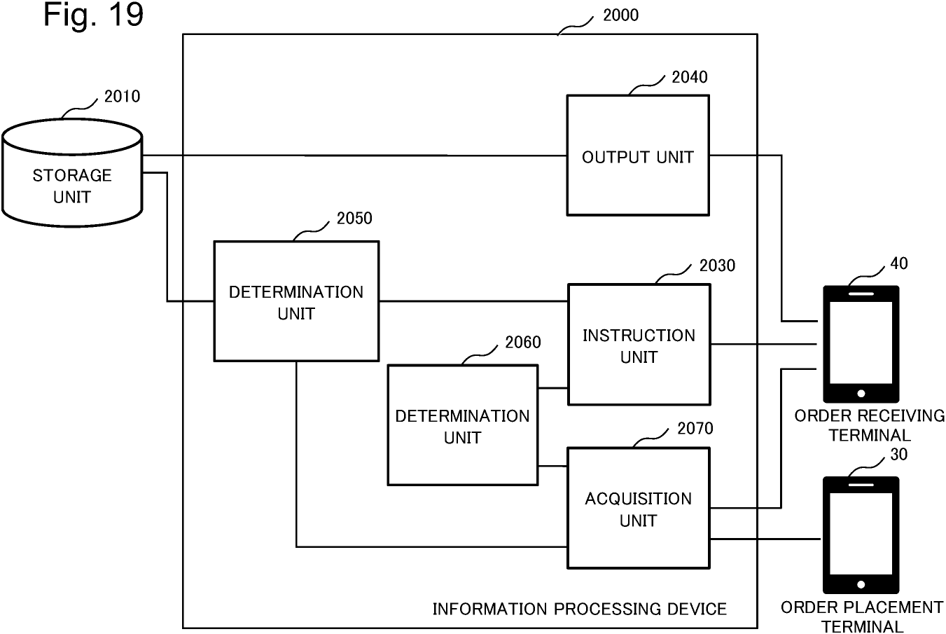

FIG. 19 is a diagram illustrating a functional configuration of the information processing device 2000 according to the second example embodiment. The information processing device 2000 according to the second example embodiment includes the acquisition unit 2020, the instruction unit 2030, the output unit 2040, the determination unit 2050, and an assessment unit 2060. The assessment unit 2060 constitutes an assessment means that assesses whether the information about a measurement environment, acquired from the acquisition unit 2020, in which the contractor can make a measurement and the measurement environment information when the object to be measured is acquired satisfy a predetermined condition.

The second example embodiment is different from the other example embodiments in that an assessment unit 2060 and an acquisition unit 2070 are included. The operations of the instruction unit 2030, the output unit 2040, and the determination unit 2050 are similar to those in the other example embodiments, and the description of the operations will be omitted in the present example embodiment.

### < Flow of Processing >

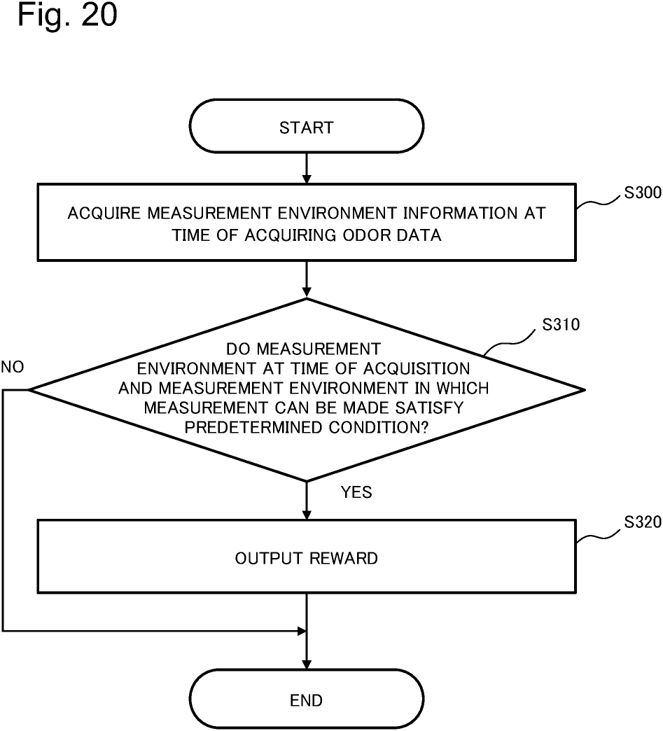

FIG. 20 is a diagram illustrating a flow of processing executed by the assessment unit 2060 included in the information processing device 2000 according to the second example embodiment. The acquisition unit 2070 acquires the measurement environment information when the odor of the object to be measured is measured and the measurement environment information desired by the orderer to output the acquired information to the assessment unit 2060 (S300). A method by which the acquisition unit 2070 acquires the measurement environment information at the time of measurement will be described later. The assessment unit 2060 assesses whether the measurement environment information at the time of measuring the odor of the object to be measured and the information about a measurement environment in which a measurement can be made satisfy a predetermined condition (S310). When the assessment unit 2060 determines that the predetermined condition is satisfied (S300: YES), the assessment unit 2060 instructs the output unit 2040 to output a reward. When the assessment unit 2060 determines that the predetermined condition is not satisfied (S300: NO), the assessment unit 2060 ends the process.

### < Method in Which Acquisition Unit 2070 Acquires Measurement Environment Information >

A method in which the acquisition unit 2070 acquires measurement environment information when the odor is measured will be described. The acquisition unit 2070 acquires temperature and humidity simultaneously measured by the sensor 10 together with odor data as measurement environment information when an odor is measured.

The method in which the acquisition unit 2070 acquires the measurement environment information is not limited to directly acquiring the temperature and humidity from the sensor 10, but may be any method as long as the actual measurement environment information can be acquired when the odor is measured. For example, the acquisition unit 2070 may use public information (weather information or the like announced by the Meteorological Agency) obtained through the weather information Internet or the like announced by the Meteorological Agency as the measurement environment information.

### < Operation of Assessment Unit 2060 >

The operation of the assessment unit 2060 will be described. The assessment unit 2060 assesses whether the measurement environment information, acquired from the acquisition unit 2070, at the time of measuring the odor of the object to be measured and the information about a measurement environment in which a measurement can be made satisfy a predetermined condition. The assessment unit 2060 outputs the determination result to instruction unit 2030.

An example of the predetermined condition used by the assessment unit 2060 will be described. The assessment unit 2060 uses, as a predetermined condition, matching between the measurement environment information when acquired and the information about a measurement environment in which a measurement can be made. Specifically, when the measurement environment information when acquired matches the information about a measurement environment in which a measurement can be made, the assessment unit 2060 determines that the predetermined condition is satisfied.

When the assessment unit 2060 assesses the matching, an error may be allowed based on a predetermined threshold value.

### < Determination Result of Assessment Unit 2060 >

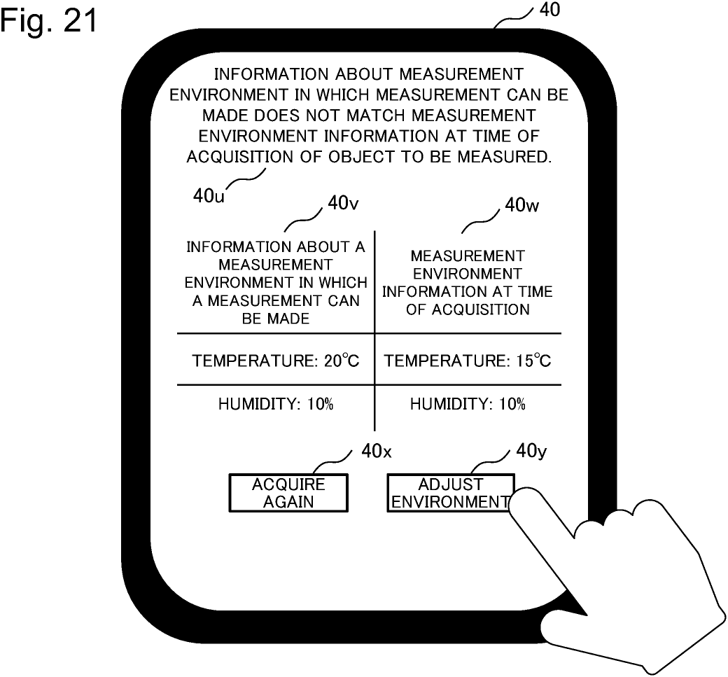

The instruction unit 2030 displays, on the order receiving terminal 40, the determination result acquired from the assessment unit 2060. Here, a determination result of the assessment unit 2060 will be described. When the determination result of the assessment unit 2060 satisfies the predetermined condition (S300: YES), the instruction unit 2030 may display the screen illustrated in FIG. 14 on the order receiving terminal 40. When the determination result of the assessment unit 2060 does not satisfy the predetermined condition (S300: NO), the instruction unit 2030 may display the screen illustrated in FIG. 21 on the order receiving terminal 40. FIG. 21 is a diagram illustrating a screen displayed by the order receiving terminal 40 when the measurement environment information at the time of measuring the odor of the object to be measured does not match the information about a measurement environment in which the contractor can make a measurement.

The screen illustrated in FIG. 21 includes a message 40u indicating that the measurement environment information at the time of measuring the odor of the object to be measured does not match the information about a measurement environment in which the contractor can make a measurement, information 40v about a measurement environment in which a measurement can be made, measurement environment information 40w at the time of measuring the odor of the object to be measured, a reacquisition button 40x, and an environment adjustment button 40y.

The information 40v about a measurement environment in which a measurement can be made is information about a measurement environment in which the contractor can make a measurement and is acquired from the order receiving terminal 40 before the acquisition of the odor data. The screen illustrated in FIG. 21 indicates “temperature: 20° C.” and “humidity: 10%” as an example of the information 40v about a measurement environment in which the contractor can make a measurement.

The measurement environment information 40w at the time of acquisition is information, acquired from the order receiving terminal 40, about a measurement environment in which a measurement can be made after the acquisition of the odor data.

The reacquisition button 40x is a button for instructing to notify the information processing device 2000 that the contractor acquires the odor data of the object to be measured again. When the contractor presses the reacquisition button 40x, the screen illustrated in FIG. 21 transitions to a screen for acquiring the odor data illustrated in FIG. 12.

The environment adjustment button 40y is a button for instructing to notify the information processing device 2000 that the contractor inputs information about a measurement environment in which a measurement can be made again. When the contractor presses the environment adjustment button 40y, the screen illustrated in FIG. 21 transitions to a screen illustrated in FIG. 7 for receiving information about a measurement environment in which the contractor can make a measurement with the sensor 10.

### < Action and Effect >

In the second example embodiment, the assessment unit 2060 compares the information, input by the contractor through the order receiving terminal 40, about a measurement environment in which a measurement can be made with the measurement environment information when the measurement is actually performed using the sensor 10, and gives an instruction of correction when the predetermined condition is not satisfied. Therefore, in the second example embodiment, the odor data in the desired measurement environment information can be accurately acquired.

### First Modification

The first modification of the second example embodiment will be described. In the second modification, the assessment unit 2060 makes a determination by using data of the odor of the object to be measured instead of measurement environment information when the odor of the object to be measured is measured.

Specifically, first, the acquisition unit 2070 acquires the object to be measured, the odor data of the object to be measured, and the measurement environment information desired by the orderer to output them to the assessment unit 2060. Then, the assessment unit 2060 acquires the odor data (2) having the same object to be measured and measurement environment as the odor data (1) input from the acquisition unit 2070 from the previously acquired odor data illustrated in FIG. 9. Then, the assessment unit 2060 calculates the feature amounts of the odor data in (1) and (2), and assesses whether a distance between the feature amounts is equal to or less than a predetermined threshold value.

Examples of the method by which the assessment unit 2060 calculates the feature amount from the odor data (1) and (2) include an average value of the odor data obtained by detecting the object to be measured a plurality of times using the sensor 10, a value indicating a feature in the shape of the detection value, and a value of a component configuration when the odor data is decomposed into exponential components, a maximum value, a minimum value, and a median value. The assessment unit 2060 may use the value of the odor data as the feature amount.

### < Action and Effect >

In the first modification of the second example embodiment, the assessment unit 2060 compares the calculated feature amount of the odor data with the feature amount of the odor data acquired in the past under the measurement environment desired by the orderer, and assesses whether the distance between the compared feature amounts is equal to or less than a predetermined threshold value. Therefore, in the first modification of the second example embodiment, odor data in desired measurement environment information can be accurately acquired.

### Third Example Embodiment

Hereinafter, the third example embodiment according to the present invention will be described.

### < Example of Functional Configuration of Information Processing Device 2000 >

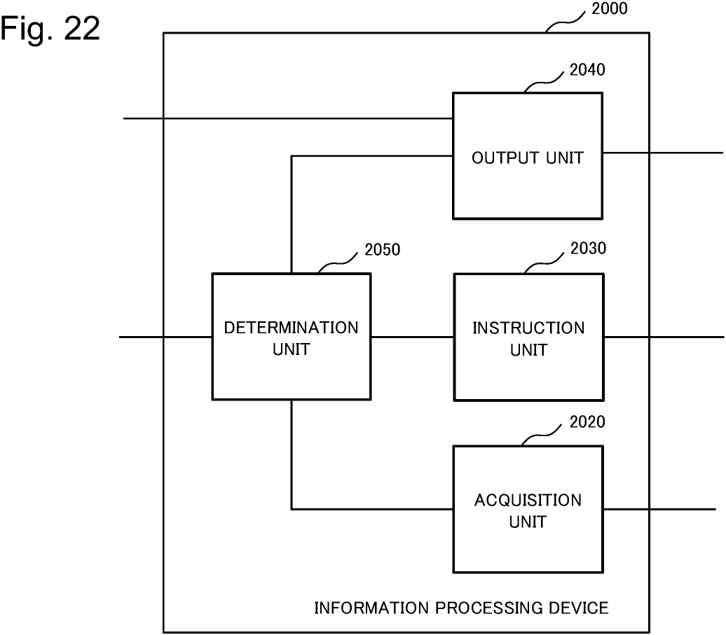

FIG. 22 is a diagram illustrating a functional configuration of the information processing device 2000 according to the third example embodiment. The information processing device 2000 includes an acquisition unit 2020, an instruction unit 2030, an output unit 2040, and a determination unit 2050. The acquisition unit 2020 acquires the object to be measured and the measurement environment information desired by the orderer to output the object to be measured and the measurement environment information to the determination unit 2050. The acquisition unit 2020 acquires information about a measurement environment in which the measurer can make a measurement with the sensor 10 to output the measurement environment information to the determination unit 2050. The determination unit 2050 determines an object to be measured that the measurer is instructed to measure based on the desired object to be measured and measurement environment information input from the acquisition unit 2020 and the information about a measurement environment in which a measurement can be made. The instruction unit 2030 instructs the measurer to measure the determined object to be measured. The output unit 2040 outputs a reward to the measurer who has acquired the odor data of the object to be measured.

### < Action and Effect >

The third example embodiment can perform crowdsourcing on odor data in consideration of the above-described characteristics.

The invention of the present application is not limited to the above-described example embodiment as it is, and can be embodied by modifying the constituent elements in a range not departing from the gist thereof at the implementation stage. Various inventions can be formed by appropriately combining a plurality of constituent elements disclosed in the above example embodiment. For example, some components may be deleted from all the components illustrated in the example embodiments. Furthermore, the components of different example embodiments may be appropriately combined.

### REFERENCE SIGNS LIST

10 sensor
13 object to be measured
14 object other than object to be measured
20 time series data
30 order placement terminal
30a object to be measured selection field
30b measurement environment information (temperature) selection field
30c measurement environment information (humidity) selection field
30d order button
40 order receiving terminal
40a message indicating that information about measurement environment in which contractor can make measurement with odor sensor is output
40b information about measurement environment in which contractor can make measurement with odor sensor
40c output button
40d message indicating that information about measurement environment in which contractor can make measurement with odor sensor is output
40e object to be measured
40f reward
40g order acceptance button
40h message indicating that odor data is being acquired
40i message indicating completion of acquisition of odor data
40j output button
40k message to display reward for acquired odor data
401 reward
40m end button
40n additional order acceptance button
40o message to instruct capturing of measurement environment
40p message to select ordered item
40q orderable item
40r message indicating that information about measurement environment in which contractor can make measurement with odor sensor is output
40s change condition
40t completion button
40u message indicating that measurement environment information when acquired does not match information about measurement environment in which contractor can make measurement with odor sensor
40v information about measurement environment in which measurement can be made
40w measurement environment information when acquired
40x reacquisition button
40y environment adjustment button
100 information processing system
1000 calculator
1020 bus
1040 processor
1060 memory
1080 storage device
1100 input/output interface
1120 network interface
2000 information processing device
2010 storage unit
2020 acquisition unit
2030 instruction unit
2040 output unit
2050 determination unit
2060 assessment unit
2070 acquisition unit

What is claimed is:

1. An information processing device comprising:
at least one memory storing instructions; and
at least one processor configured to access the at least one memory and execute the instructions to:
acquire an object to be measured and measurement environment information and information about a measurement environment in which a measurer is allowed to make a measurement with an odor sensor;
determine, based on the object to be measured and the measurement environment information and the measurement-allowed measurement environment information, an object to be measured that the measurer is instructed to measure;
instruct the measurer to measure the determined object to be measured; and
output a reward to the measurer after an acquisition of odor data of the instructed object to be measured.

2. The information processing device according to claim 1, wherein
the at least one processor is further configured to execute the instructions to:
determine whether the measurement environment information and measurement environment information at a time of measuring the object to be measured satisfy a predetermined condition; and
output the reward when the condition is satisfied.

3. The information processing device according to claim 1, wherein
the at least one processor is further configured to execute the instructions to:
instruct a change in a measurement environment; and
output a reward to the measurer when odor data of the instructed object to be measured in information about the changed measurement environment is acquired.

4. The information processing device according to claim 1, wherein
the at least one processor is further configured to execute the instructions to:
acquire an image including the measurable object to be measured, and wherein
determine an object to be measured that the measurer is instructed to measure based on the object to be measured and the measurement environment information, and the image and the measurement-allowed measurement environment information.

5. The information processing device according to claim 1, wherein
store odor data and measurement environment information when the odor data is acquired; and
determine the reward based on a statistic of the measurement environment information when the odor data is acquired.

6. An information processing method comprising:
acquiring an object to be measured and measurement environment information and information about a measurement environment in which a measurer is allowed to make a measurement with an odor sensor;
determining, based on the object to be measured and the measurement environment information and the measurement-allowed measurement environment information, an object to be measured that the measurer is instructed to measure;
instructing the measurer to measure the determined object to be measured; and
outputting a reward to the measurer when odor data of the instructed object to be measured is acquired.

7. A non-transitory storage medium storing a program for causing a computer to execute:
a step of acquiring an object to be measured and measurement environment information and information about a measurement environment in which a measurer is allowed to make a measurement with an odor sensor;
a step of determining, based on the object to be measured and the measurement environment information and the measurement-allowed measurement environment information, an object to be measured that the measurer is instructed to measure;
a step of instructing the measurer to measure the determined object to be measured; and
a step of outputting a reward to the measurer when odor data of the instructed object to be measured is acquired.
